(19) United States
(12) Patent Application Publication
(10) Pub. No.: US 2024/0398293 A1
(43) Pub. Date: Dec. 5, 2024

(54) Method and Apparatus for Non-Invasive Hemoglobin Level Prediction

(71) Applicant: Marquette University, Milwaukee, WI (US)

(72) Inventors: Md Kamrul Hasan, Milwaukee, WI (US); Sheikh Iqbal Ahamed, Fox Point, WI (US); Richard R. Love, Madison, WI (US)

(21) Appl. No.: 18/806,004

(22) Filed: Aug. 15, 2024

Related U.S. Application Data
(63) Continuation of application No. 16/978,129, filed on Sep. 3, 2020, now Pat. No. 12,089,930.
(60) Provisional application No. 62/638,630, filed on Mar. 5, 2018.

(51) Int. Cl. A61B 5/1455; A61B 5/00

(52) U.S. Cl. CPC A61B 5/14552; A61B 5/6826; A61B 5/6898; A61B 2576/02

(57) ABSTRACT

An image-based hemoglobin estimation tool for measuring hemoglobin can be embedded in handheld devices such as smartphones, and similar known and to be developed technology. The hand-held device acquires video data of a finger illuminated from the dorsal surface by a first near infrared light responsive to hemoglobin and a second near infrared light near responsive to plasma. The acquired video is segmented into frames and processed to produce a Photoplethysmography (PPG) waveform. The features of the PPG waveform can then be identified, and the waveform and corresponding features evaluated by a predictive hemoglobin model. The predictive hemoglobin model can be provided at a remote computer, enabling non-invasive hemoglobin analysis from point of care locations. Near infrared lights of 850 nm and 1070 nm are particularly effective in the process.

### CROSS-REFERENCE TO RELATED APPLICATIONS

This application is a continuation of U.S. patent application Ser. No. 16/978,129 filed on Sep. 3, 2020, which represents the U.S. national stage entry of International Application No. PCT/US2019/020675 filed on Mar. 5, 2019, which claims the benefit of U.S. Provisional Patent Application No. 62/638,630 filed on Mar. 5, 2018, which disclosures are incorporated herein by reference in their entirety.

### BACKGROUND

Hematologic diseases are a major global public health problem. The principal constituent of blood is hemoglobin in red blood cells. Broadly, hematologic diseases are of two major types: the anemias and hematologic disorders—primarily hemoglobinopathies. Hemoglobin functions to carry oxygen to body tissues, which activity is compromised with disease. Iron-deficiency anemia occurs in 2 billion people worldwide, and during the past decade, the numbers of people affected has increased. The World Health Organization (WHO) has estimated that anemia affects about 25% of the global population, and an average of 5.6% of the US population. Anemia is particularly problematic in children, because it enhances the risk of cognitive development impairment, and in pregnant women, who suffer higher maternal mortality rates. Many of those suffering from anemia are in developing countries where medical resources are limited.

The most common hematological disorder is sickle cell hemoglobinopathy, called sickle cell disease (SCD). SCD patients are anemic and have abnormal, sickle-shaped red blood cells, the percentages of which increase under stress (such as with infections) causing small vessel obstruction. The most common clinical problems with SCD patient are crises with acute and sever musculoskeletal pain. In the United States, according to the Centers for Disease Control and Prevention (CDC), about 100,000 Americans have SCD and the cases numbers are increasing. Approximately one in 365 African Americans and one in 16,300 Hispanic Americans have SCD.

Currently, the most common measure to assess for hematologic disease is a laboratory plasma hemoglobin (Hgb) test, which determines the concentration of hemoglobin in the blood. These laboratory tests are done on venous or capillary blood specimens obtained invasively, most commonly with drawing blood from a vein, which involves insertion of a needle. Patients therefore can feel discomfort, pain, numbness, or a shocking sensation. Itching or burning at the collection site is also common. These procedures can be particularly traumatic for children and mentally disabled persons. Additionally, these tests require travel to a medical facility, and can be expensive. While there are some point-of-care systems for hemoglobin assess, these are also expensive. In sum, the current technology is inconvenient, costly, slow, uncomfortable and for many not readily accessible.

Some non-invasive point-of-care tools for assessment of hemoglobin levels are available. However, these tools are expensive, have poor performance measures, and require specific training for proper operation and appropriate use. As a result, only large research centers and hospitals can purchase, operate, and maintain these systems.

Recently, smartphone-based hemoglobin measurement technologies have been developed for hemoglobin level assessment. Some of these technologies rely on analysis of the lower eyelid conjunctiva, which has been shown to be useful because the conjunctival mucosa is thin and the underlying micro-vessels are easily seen. One such smartphone-based system compares conjunctival pallor with an eye color chart. Estimation of precise hemoglobin levels with these systems is presently poor.

In these circumstances, a non-invasive, easy-to-use, inexpensive measure of hemoglobin levels is desirable to improve access to diagnostics and to provide safe management of patients with hematologic disease.

### SUMMARY

In one aspect, the present disclosure provides a method for non-invasively blood hemoglobin levels. The method comprises acquiring a time-based series of images of the finger ventral pad-tip illuminated from the dorsal side of the finger with a near infrared light responsive to blood hemoglobin, and white light, and acquiring a second time-based series of images of the finger ventral pad-tip illuminated from the dorsal side of the finger with a near infrared light responsive to blood plasma, and white light. Each image in each of the first and second time-based series is divided into groups of blocks. A time series signal is generated from each block, and at least one Photoplethysmography (PPG) cycle is identified from each of the time series signals, including a systolic peak and a diastolic peak. The PPG cycles are processed to determine blood hemoglobin levels.

The step of acquiring a time-based series of images can include acquiring a first and a second video. The video can be separated into frames, each frame comprising an image.

The near infrared light responsive to blood hemoglobin can have a wavelength of between 800 and 950 nm, and the near infrared light responsive to plasma can have a wavelength of 1070 nm. The near infrared light responsive to blood hemoglobin can have a wavelength of 850 nm.

The method can include calculating a ratio of the PPG signal of the first time-based series of images of a blood flow illuminated with a near infrared light responsive to blood hemoglobin, to the second time-based series of the images of a blood flow illuminated with a near infrared light responsive to blood plasma.

The method can also comprise identifying at least one feature in each of the PPG cycles, and the feature can be used to determine the hemoglobin level. The feature can comprise at least one of a relative augmentation of a PPG, an area under the systolic peak; an area under a diastolic peak, a slope of the systolic peak, a slope of the diastolic peak, a relative timestamp value of the peak, a normalized PPG rise time, a pulse transit time (PTT), a pulse shape, or an amplitude.

The step of processing the PPG can comprise analyzing the PPG signals using a prediction model constructed using a support vector machine regression.

The step of generating a time series signal for each of the first and second time-based series of images comprises acquiring red green blue (RGB) digital images of a blood flow. Here, the step of subdividing each image into a plurality of blocks further comprises subdividing each image into a plurality of blocks further comprising a defined number of pixels, calculating a mean intensity value for the red pixels in each block, generating the time series signal identifying each image in the series versus an average value of a block, and subsequently identifying at least one PPG signal in each time series.

In another aspect, a system for non-invasive analysis of a hemoglobin level is disclosed. The system comprises a camera, a first lighting device comprising a near infrared light of a wavelength responsive to blood hemoglobin and adapted to provide images of a finger of a subject, and a second lighting device comprising a near infrared light of wavelength responsive to blood plasma and adapted to provide images of a finger of a subject, and at least one processor. The processor is programmed to receive a first time series of images of a finger of a subject while illuminated by the first lighting device, the first time series of images acquired under conditions selected to capture at least one complete detailed Photoplethysmography (PPG) cycle representative of blood hemoglobin and to receive a second time series of images of the finger while illuminated by the second lighting device, the second time series of images acquired under conditions to capture at least one complete detailed PPG cycle representative of plasma. The processor is further programmed to identify at least one feature in the PPG cycle representative of blood hemoglobin, identify at least one feature in the PPG cycle representative of blood plasma, and provide the identified feature representative of blood hemoglobin and the feature representative of blood plasma to a predictive model adapted to identify a hemoglobin level as a function of the features.

The processor can be further programmed to calculate a ratio of the at least one feature in the PPG cycle representative of blood hemoglobin to the at least one feature in the PPG cycle representative of blood plasma and provide the ratio to a predictive model adapted to identify a hemoglobin level as a function of the ratio.

The camera can be a red green blue (RGB) digital camera, and, for each of the first and second time series of images, the processor can further be programmed to subdivide each image into a plurality of blocks comprising a defined number of pixels, calculate a mean intensity value for the red pixels in each block, generate a time series signal identifying each image in the series versus an average value of a block for each of the first and second time series, and subsequently identify the at least one PPG signal in each of the first and second time series.

The predictive model can be stored in a remote computer having a second processor, and the operator transmits the videos to the remote computer. The predictive model can comprise a plurality of predictive models, each corresponding to a near infrared light selected to have a wavelength responsive to blood hemoglobin.

The lighting device can comprise a plurality of light emitting diodes mounted in an enclosure, wherein the enclosure includes a slot sized and dimensioned to receive a finger for illumination. The light emitting diodes can include at least one white light LED. The enclosure comprises a material selected to minimize interference from ambient light. The lighting device can comprise one or more coupling device for coupling the lighting device to a camera.

These and other aspects of the invention will become apparent from the following description. In the description, reference is made to the accompanying drawings which form a part hereof, and in which there is shown a preferred embodiment of the invention. Such embodiment does not necessarily represent the full scope of the invention and reference is made, therefore, to the claims herein for interpreting the scope of the invention.

### BRIEF DESCRIPTION OF THE DRAWINGS

FIG. 1 illustrates a finger where its approximately 15 mm thickness is penetrated by near infra-red (NIR) light that is minimally absorbed by the intervening tissues, from the dorsal to the ventral surfaces. Through ballistic, snake and diffuse photon scattered paths, NIR light exposure on the dorsal side of the finger, despite intervening fingernail and osseous tissues, can be detected on the ventral surface.

FIG. 2 illustrates the optical densities of the responses of oxygenated hemoglobin, deoxygenated hemoglobin, and plasma illuminated by light of various wavelengths;

FIG. 3A illustrates the process of capturing a fingertip video using 850 nm NIR LED light board, and a plot of light intensity versus time (frame) where the graph defines a photoplethysmogram (PPG) signal caused by the modulation of light intensity by the changes in arterial blood volume change with each heartbeat;

FIG. 3B illustrates the process of capturing a fingertip video using 1070 nm NIR LED light board, and a plot of light intensity versus time (frame) where the graph defines a PPG signal caused by the modulation of light intensity by the changes in arterial blood volume change with each heartbeat;

FIG. 4 illustrates a PPG signal generated from a fingertip video.

FIG. 5 illustrates the ratio of two PPG signals captured under two different wavelengths of NIR.

FIG. 6 illustrates the subdivision of an image frame to generate multiple time series signals;

FIG. 7 illustrates PPG signal generation from a time series signal;

FIG. 8 illustrates multiple features of a PPG signal;

FIG. 9 illustrates feature generation from a PPG signal generated in all blocks;

FIG. 10 illustrates features captured from 100 blocks averaged.

FIG. 11 illustrates the Support Vector Machine (SVM) algorithm for linear and nonlinear regression.

FIG. 12A illustrates the regression line developed based on gold standard-laboratory-measured hemoglobin levels and the estimated hemoglobin values using Model;

FIG. 12B illustrates the Bland-Altman plot for estimated hemoglobin levels using Model;

FIG. 13 is a block diagram of a system for performing a non-invasive hemoglobin level test in accordance with at least some embodiments of the current disclosure.

FIG. 14 is a schematic of a light source device constructed in accordance with the disclosure.

### DETAILED DESCRIPTION

The present disclosure relates to the measurement of blood hemoglobin concentration using two optical absorption video sets of signals captured under near infrared light exposure with pulsatile blood volume changes. The blood volume changes are captured in the photoplethysmogram (PPG) signals generated. As described below, the measurement can be performed using a hand-held computing device such as a cell phone or smartphone. Images of dorsal fingertip tissue exposed to near infrared light wavelengths selected based on responsiveness to plasma and hemoglobin are acquired with simultaneous dorsal fingertip exposure to white light. The images can, for example, be obtained as a 10 second video of the ventral finger pad. The images allow creation two sets of photoplethysmogram (PPG) signal features that can be analyzed together to determine blood hemoglobin levels.

### Photoplethysmogram

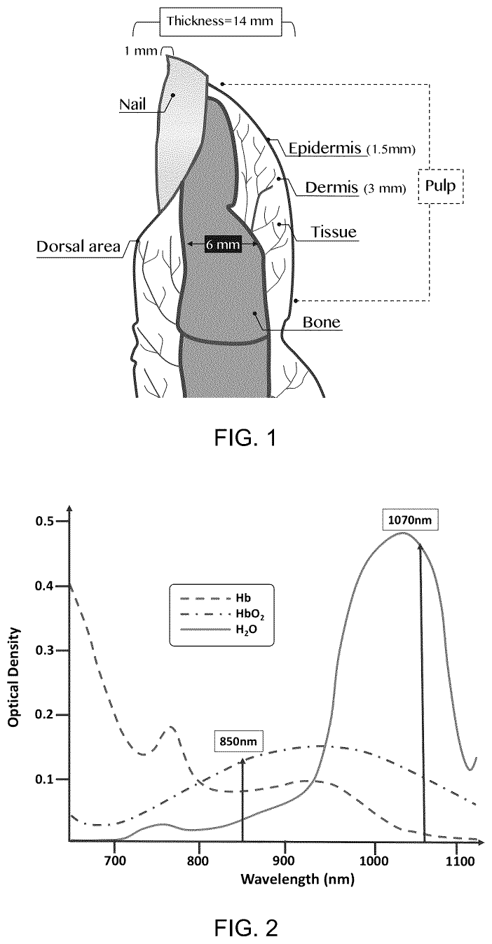

PPG is an optical technique for observing blood volume changes noninvasively in the microvascular bed of tissue. Referring now to FIG. 1, a PPG system includes a light source and a photodetector where the light source illuminates the tissue area (e.g., a finger), and the photodetector captures the variation of light intensity. In IR or near-IR wavelengths, the changes in blood flow in tissues such as finger and muscle due to arteries and arterioles can be detected using PPG sensors. The PPG signal can be captured by detecting light intensity which is reflected or transmitted from the tissue. The intensity variations are observed due to vascular blood pressure changes. The PPG signal represents the differences in light intensities with the pulse.

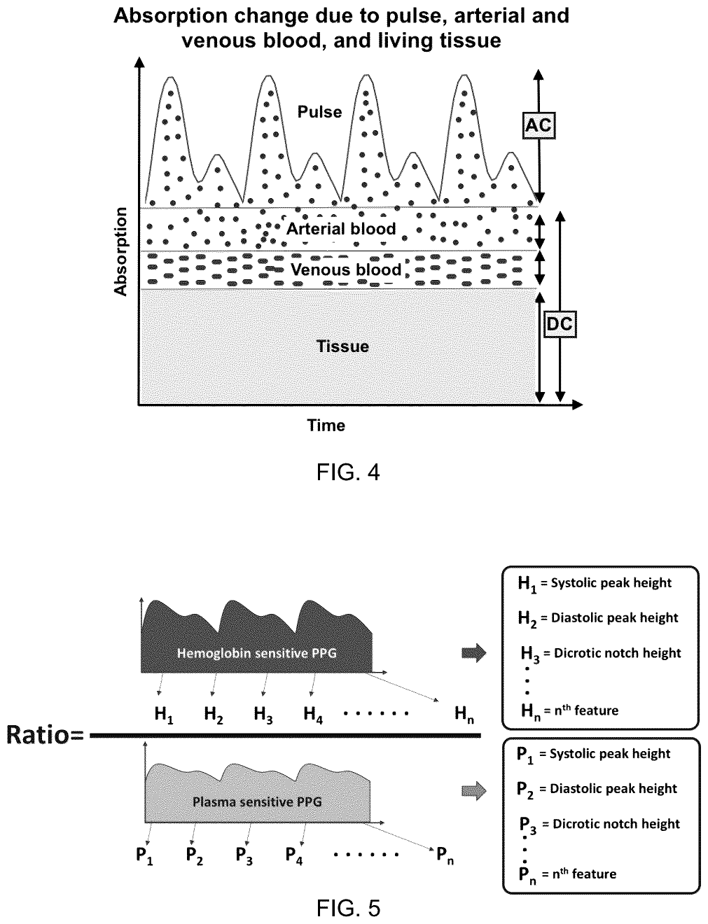

A PPG waveform has two main components: a direct current (DC) component and an alternating current (AC) component. The direct current (DC) component is generated by the transmitted or reflected signal from the tissue and the average blood volume of both arterial and venous blood (see FIG. 4). The AC component fluctuates with the blood volume in the systolic and diastolic phases. When a finger is illuminated under two different wavelengths of NIR lights, and a ratio between the AC and DC components is determined for each, the effects from tissue and venous blood can be removed, providing a measure of the hemoglobin level.

To measure the hemoglobin level with respect to the blood plasma level, one response can be from the blood hemoglobin and another response from the blood plasma. In living tissue, water absorbs photons strongly above the 1000 nm wavelength of light; melanin absorbs in the 400 nm-650 nm spectrum. Hemoglobin response occurs across a spectrum from 650 to 950 nm. The spectrum range from 650 nm to 1100 nm is known as the tissue optical window or NIR region. To get a response from hemoglobin, an 850 nm wavelength NIR LED light which is hemoglobin responsive can be used. Similarly, to get a response from blood plasma, a 1070 nm wavelength NIR LED that is blood plasma responsive can be used. By analyzing the ratio of these two responses as presented as PPG signals, the tissue absorbance effects are removed and a more detailed characteristic of a PPG signal can be obtained for hemoglobin and plasma.

Referring now to FIG. 2, in the finger, the blood, tissue, and bone absorb much of the non-IR (or visible range) light. A video camera can be used to capture the transmitted light, which changes based on the pulsation of arterial blood. The pulsation response can be extracted in time series data calculated from the fingertip video and converted into a PPG signal, which can be analyzed to build a hemoglobin prediction model. A small lighting surface can penetrate only a small part of the living tissue whereas a large planar lighting surface enables penetration of light to a deeper level (such as around bone tissue).

### Acquire Image Data for a PPG Signal

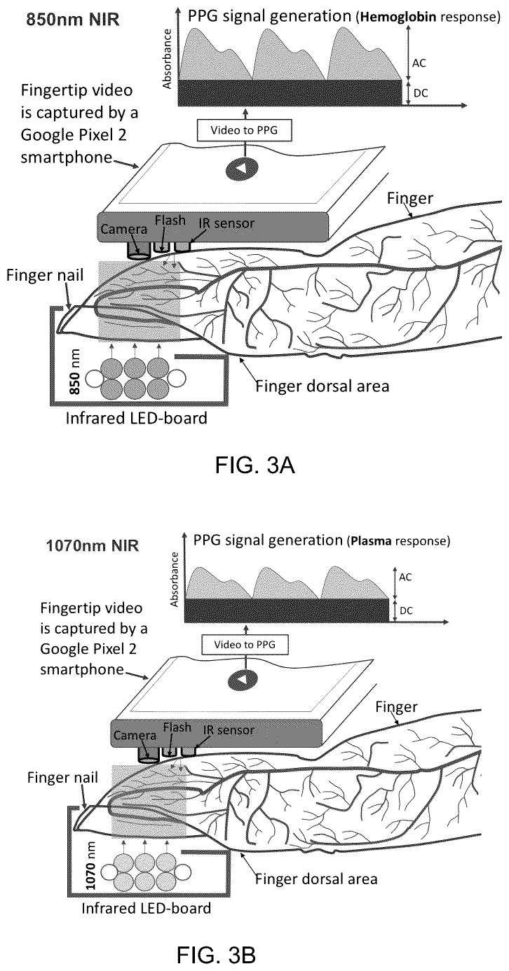

FIGS. 3A and 3B illustrate the approach for acquiring data. A finger, such as the index finger, is illuminated by two near-infrared (NIR) light sources with unequal wavelengths λ_{H} and λ_{P}. The wavelength λ_{H} is substantially sensitive to hemoglobin and insensitive to any other blood component. The light of wavelength λ_{P} provides a significant response to blood plasma where other blood constituents have no response or negligible response under this NIR (λ_{P}) light. Here, 850 nm as λ_{H} and 1070 nm as λ_{P} NIR LED lights are used. To increase the amount of surface area that is illuminated, a number of LEDs of the same wavelength can be used. In our system, six 850 nm NIR and two white LED lights were used for the hemoglobin response (light source L₈₅₀, having a wavelength λ_{H}), and six 1070 nm NIR, and two LED white lights were used for the plasma response (light source L₁₀₇₀, having wavelength λ_{P}). The NIR and white light are always turned on while collecting the data. The white light enables acquiring a photo of the finger that can be visualized.

Referring still to FIGS. 3A and 3B, the light beams of both the L850 nm and L1070 nm light sources are applied to cross from the dorsal side of the finger to the pulp area, resulting in scattering and absorption in the tissue and bone. The light beams exit the ventral pad side of the finger by transmission and transflection and are captured by a video camera. By placing two different light sources L₈₅₀ and L₁₀₇₀ under the dorsal side of a finger at different times, the response of hemoglobin and plasma can be captured in the fingertip videos, and these videos can then be converted to PPG signals. Here, one PPG signal is extracted from a video captured using light source L₈₅₀ and another PPG signal is generated using the fingertip video recorded under L₁₀₇₀. Both PPG signals are presented in FIG. 3. A plot of the PPG intensity received for one light source over time or across the frame number is illustrated in FIG. 4. The relative magnitude of the AC signal is due to increased amount of blood (in systolic phase) and the decreasing amount of blood (in the diastolic phase). In addition to the AC component, there is a DC component that is steady in magnitude since this light intensity is captured in the tissue and non-pulsating venous blood. The value of each PPG signal captured for both light sources L₈₅₀ and L₁₀₇₀ are normalized by dividing the AC component by the DC component. Here, the value calculated by AC₈₅₀/DC₈₅₀ is defined as R₈₅₀ and AC₁₀₇₀/DC₁₀₇₀ as R₁₀₇₀. The normalized value of a PPG signal cancels out the effect of tissue, so that R₈₅₀ represents the hemoglobin response and R₁₀₇₀ the plasma response. By calculating the ratio of R₈₅₀ and R₁₀₇₀, a relationship is generated which provides the information on the light absorbed by both hemoglobin and plasma. The ratio of R₈₅₀ and R₁₀₇₀ for each subjects' PPG signal in a mathematical model is then highly correlated with laboratory-measured (“gold standard”) hemoglobin values as shown in FIG. 5. In addition to the ratio of AC and DC component of a PPG, other features from the PPG signal such as relative augmentation of a PPG, area under the systolic peak and diastolic peak, a slope of each peak, and a relative timestamp value of the peak, can be calculated or otherwise determined, as discussed below.

### Pre-Process Data and Identify Region of Interest in Images

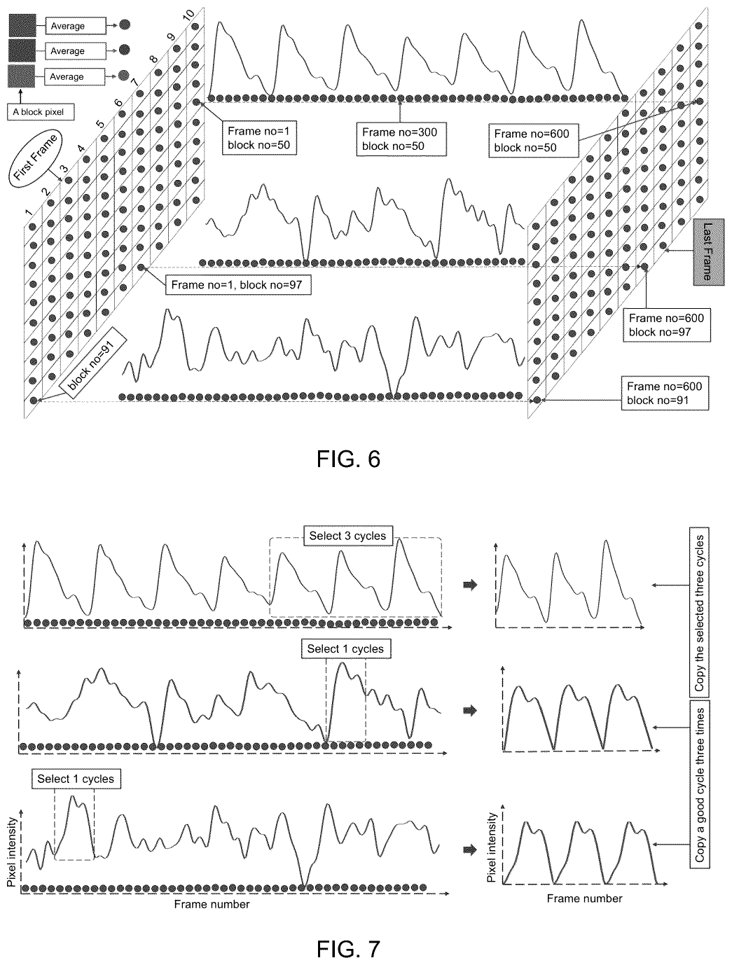

To identify a region of interest in the acquired video data, the following steps are taken:
1. Extract all frames from the video.
2. Subdivide each frame into blocks and assign an index number to each block. In one example, the frames were divided into 10×10 blocks, and the index numbers ranged from 1 to 100 where the number 1 starts from top left part of the frame increases towards the right (See FIG. 6).
3. Generate time series signal for each block from the starting frame to the last frame of the video
4. For each time series signal, perform the following steps:
   a. Apply bandpass filter to filter noise from the acquired video. In one example, a bandpass filter of 0.66 Hz-8.33 Hz was used, where the minimum cut off value was selected to discard the signal fluctuations due to breathing (0.2-0.3 Hz).
   The other sources of noise can include finger movements, finger quaking resulting in motion artifacts, coughing, and gasping.
   b. Sample using the Nyquist frequency as frames per second (FPS)/2. In one example, the frames per second is 60, and FPS/2 is 60/2=30.
   c. Filter the data to remove areas of fluctuation at the beginning and end due to finger movement to start and stop the video camera.
   d. Define this filtered and cropped signal as the PPG signal and look for three good PPG cycles where each cycle includes a systolic peak and a diastolic peak.
   e. If three continuous PPG cycles are not found, then select at least one cycle which has a systolic peak and a diastolic peak, replicate the selected cycle three times, and combined them to make a three-cycle PPG signal as shown in FIG. 7.
   f. Transfer this PPG signal with three cycles to extract the features.

Referring now to FIG. 6, in one example, six 140 mW NIR LEDs were used, along with two white LED lights. These eight LED lights were put in one LED-board which was used for video recording. Three LED boards were created with three light wave-lengths: 850 nm, 940 nm, and 1070 nm NIR LED lights. Videos were acquired at a rate of 60 frames per second (FPS), by a camera that had a 1080×1920-pixel resolution. Here, in a 10-second video, there are 600 frames per 10 second video, and a single block of 10×10 block matrix contains 108×192 pixels of information.

Referring still to FIG. 6, each frame of the video has three two-dimensional pixel intensity arrays for each color: red, green, and blue (RGB). Since each frame has 10×10 blocks, a mean value is computed from each color pixel for each block of a frame which gives 100 mean values (dots in FIG. 6) for one frame. In FIG. 6, 600 frames extracted from a fingertip video are illustrated as subdivided into the 10×10 block matrix. Then, a time series signal is generated, with the frame number in the X-axis and the calculated averaged value of a block in Y-axis. FIG. 6 illustrates three different time series signals for red pixel intensity between first and last frame where the top signal was generated by block number 50, the middle signal was made by block number 97, and the third signal was calculated from block number 91. The dot in each block represents the average of all red pixel intensities of the block area. This dot is the averaged value of the all red pixels in the block. Since each dot has a different intensity, the plot of their averaged values across all frames produce a time series signal. Only red pixel intensities were used because only weak intensity signals were found with green and blue pixels.

After generating the PPG signal from the fingertip video, features were extracted from each PPG signal. Referring now to FIG. 7, three PPG cycles for each block of a video were captured. From these, the AC (systolic peak) and DC (trough) can be measured and used for hemoglobin level analysis.

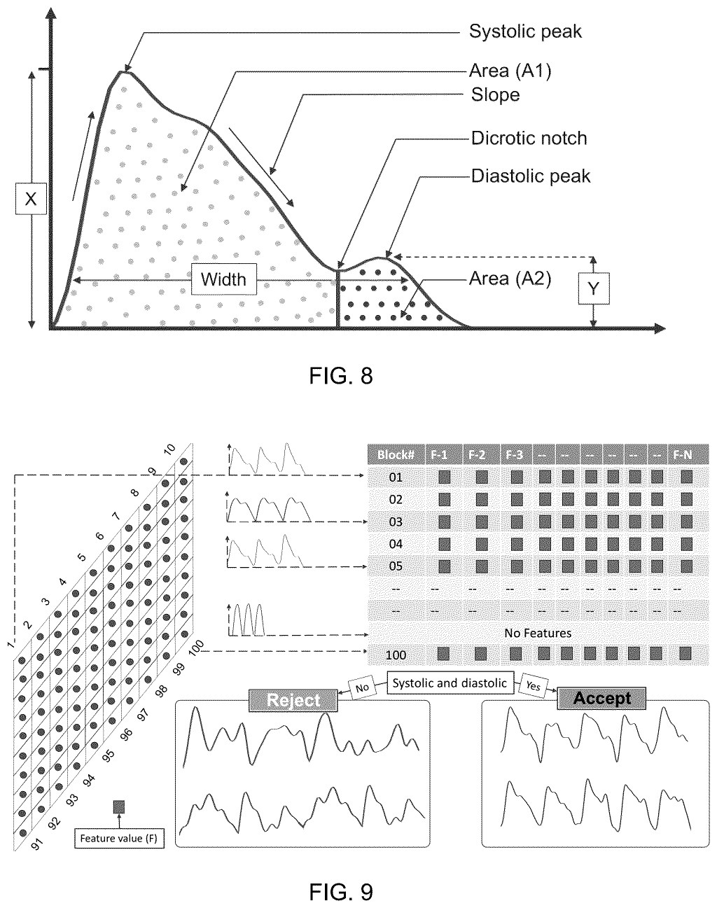

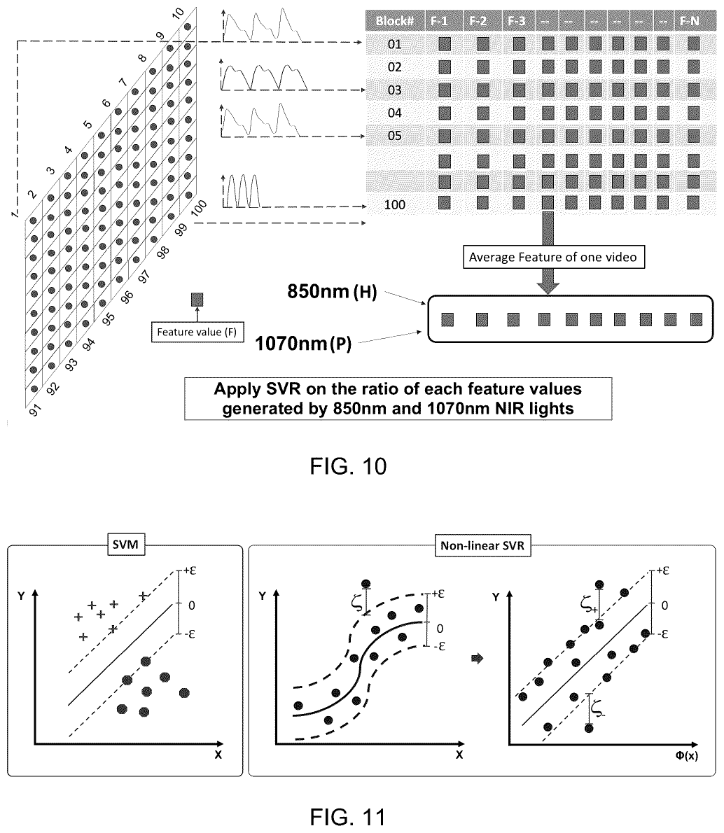

Referring now to FIG. 8, to characterize the PPG signal generated on each infrared (IR) LED light more fully, features including its diastolic peak, dicrotic notch height, ratio and augmented ratio among systolic, diastolic, and dicrotic notch, systolic and diastolic rising slope, and inflection point area ratio were extracted. About 80% of blocks that have a PPG include these features. The rest of the blocks are assigned as no feature values as shown in FIG. 9 and filtered out. To determine whether a specific PPG signal should be used, systolic and diastolic peaks are noted, and the height of the systolic peak is checked to verify that it is higher than the diastolic peak. If any block has no single PPG cycle that satisfies the selection criteria, the signal does not provide an adequate PPG, and the features are not determined. Finally, the PPG features calculated from a fingertip video are averaged (See FIG. 10).

### Constructing the Model

To develop a hemoglobin prediction model, fingertip videos and corresponding known gold standard hemoglobin levels of 167 adult individuals were used; these data were selected from an initial set from 212 individuals. Forty-five cases exhibited poor quality video images or missing laboratory values and were filtered out. Of the remaining 167 subjects, 82 were men and 85 were women. Laboratory hemoglobin levels ranged from 9.0-13.5 gm/dL across the set of subjects. Video data were acquired with the finger illuminated with three LED boards at 850 nm, 940 nm, and 1070 nm light wave lengths. The data were analyzed using the Support Vector Machine Regression (SVR), where SVR uses “Gaussian” kernels to build the prediction model using support vectors.

The Support Vector Machine (SVM) maximizes the boundary value (sometimes called a “wide street”) to generate a line that separates two classes, as illustrated in FIG. 11. In the regression, the model predicts a real number and optimizes the generalization bounds given for regression. Here, the loss function is known as the epsilon intensive loss function as shown in FIG. 11. In SVR, the input matrix is mapped onto multi-dimensional feature space applying nonlinear mapping to build a linear model as shown in Equation 1 where φj(x), j=1, 2, 3, m is a set of nonlinear transformations and ‘b’ is the ‘bias’ term.

$$f ( x , ω ) = {\sum }_{j = 1}^{m} {ω}_{j} {φ}_{j} ( x ) + b \qquad ( 1 )$$

The SVR uses ε-intensive loss function.

$$min \frac{1}{2} {‖ ω ‖}^{2} + C \sum ( {ζ}_{+} + {ζ}_{-} ) \qquad ( 2 )$$

subject to

$$\{ \begin{matrix}{y}_{i} - f ( {x}_{i} , ω ) \leq ∈ + {ζ}_{+} \\ f ( {x}_{i} , ω ) - {y}_{i} \leq ∈ + {ζ}_{-} \\ {ζ}_{+} , {ζ}_{-} > 0 , i = 1 , 2 , 3 … , n\end{matrix} \qquad ( 3 )$$

In the data analysis, MATLAB command “fitrsvm” was used with Xtrain, Ytrain, and “Gaussian” kernel as parameters. The “Standardize” function was set to standardize the data using the same mean and standard deviation in each data column. The prediction model was generated as a “Gaussian SVR Model” and the test data applied on this model using the MATLAB command “predict”, while providing the model and test data as the parameter. The results are illustrated using MAPE, correlation coefficient (R), and Bland-Altman plot.

The Mean Absolute Percent Error (MAPE) is a commonly used metric to present the error level in the data. The MAPE is calculated as the following equation 4.

$$M = \frac{100 \%}{1} {\sum }_{i = 1}^{n} \frac{\vert {A}_{t} - {E}_{t} \vert}{\vert {A}_{t} \vert} \qquad ( 4 )$$

Where, Aₜ=Actual value or gold standard measurement, Eₜ=estimated value, and n=number of measurements or observations. MAPE has been used because MAPE does not depend on scale.

The correlation coefficient R can also be used to determine how strongly two measurement methods are related. R is computed as the ratio of covariance between the variables to the product of their standard deviations. The value of R is in between −1.0 and +1.0. If the value of R is +1.0 or −1.0, then a strong linear relationship between two estimation methods, and the linear regression can be calculated. The R value, however, does not identify whether there is a good agreement between the measurement methods. The Bland-Altman plot was used to evaluate a bias between the mean differences and to assess the agreement between the two measurement processes. The formula for Pearson's correlation is:

$$R = \frac{{\sum }_{i = 1}^{n} ( {x}_{i} - x ) ( {y}_{i} - y )}{\sqrt{[ {\sum }_{i = 1}^{n} {( {x}_{i} - \overline{x} )}^{2} ] [ {\sum }_{i = 1}^{n} {( {y}_{i} - \overline{y} )}^{2} ]}} \qquad ( 5 )$$

where, n is the sample size, xᵢ, yᵢ are the individual sample points indexed with i,

$$\overline{x} = \frac{1}{2} {\sum }_{i = 1}^{n} {x}_{i}$$

is the sample mean, and

$$\overline{y} = \frac{1}{2} {\sum }_{i = 1}^{n} {y}_{i}$$

is the target mean value.

The Bland-Altman graph plot represents the difference between the two measurement methods against the mean value of the measurement. The differences between these two methods are normally plotted against the mean of the two measurements. A plotting difference against mean helps identify the relationship between measurement error and the clinically measured value.

As described above, the model was developed using data from 167 subjects, which was filtered from an initial set of data of 212 fingertip videos. (IR) LED lights were applied with wavelengths of 850 nm, 940 nm, and 1070 nm. A Google Pixel 2 smartphone was used to capture video at 60 frames per second (FPS). The Google Pixel 2 has a 950 nm LED on board, and video was also acquired using this LED.

Sixteen PPG features were computed from a block of a video (600 frames) including systolic peak, diastolic peak, a dicrotic notch, augmentation among those peaks, peaks arrival time, inflection point area ratio, and peak rising slopes. To normalize the data, a ratio of two PPG features generated from different wavelengths of light was used. The ratio of two PPG signals' feature values was calculated as follows:

$${R}_{1070} ( 850 ) = \frac{P P {G}_{850}}{P P {G}_{1070}} \qquad ( 6 )$$

The ratio of two PPG feature values here is the individual ratio between each feature value. For example, the ratio of the systolic peak value under a 1070 nm NIR light and the systolic peak value under an 850 nm NIR. Similarly, the ratio of all other features that were applied to the SVR machine learning algorithm were measured, along with ratios for the other wavelengths, referred to as herein as R₁₀₇₀(940), R₁₀₇₀(Pixel2) where:

$${R}_{1070} ( 940 ) = \frac{P P {G}_{940}}{P P {G}_{1070}} \qquad ( 7 )$$

$${R}_{1070} ( Pixel 2 ) = \frac{P P {G}_{P i x e l 2}}{P P {G}_{1070}} \qquad ( 8 )$$

Here, PPG1070 was considered as a plasma responsive PPG signal, as discussed above. The other PPG signals were chosen as hemoglobin responsive PPG signal.

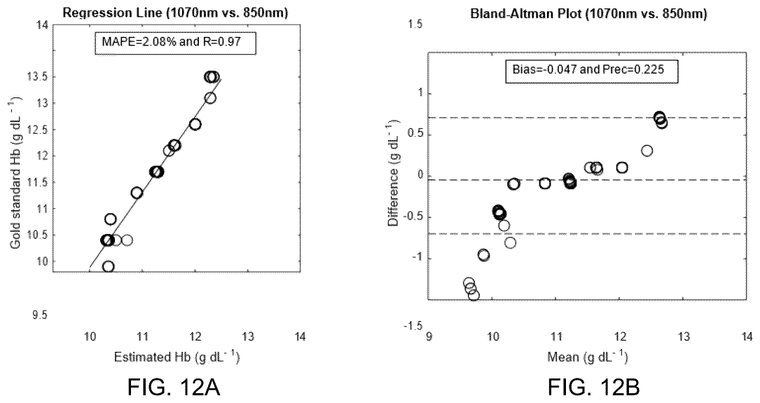

As described above, SVR was applied to the features generated from each of these ratios. For the ratio R₁₀₇₀(850) (Equation 6), an optimal prediction model was developed and defined. A regression line based on the clinically measured hemoglobin levels and the estimated hemoglobin values is illustrated in FIG. 12 based a combination of features that gave this optimal result. In FIG. 12a, the Mean Absolute Percentage Error (MAPE) is 2.08% where the linear correlation coefficient (R) between gold standard and estimated hemoglobin was 0.97.

### Comparative Predictive Model Results

Other models using data obtained with the LED light board at 940 nm, and a cell phone camera using only the white light with this phone on the ventral finger pad were developed and evaluated. The described model was found to be the most accurate and predictive.

### Hemoglobin Estimation Procedure Using the Predictive Model

With further confirmatory data, the predictive model described above can therefore be used to provide a noninvasive point of care tool for hemoglobin assessment. In this framework, a fingertip video is recorded while the finger is illuminated by two near-infrared (NIR) light sources with unequal wavelengths, one that is sensitive to hemoglobin (λ_{H}) and another that is sensitive to plasma (λ_{P}). The videos are then processed as described above and analyzed as in the defined optimal prediction model.

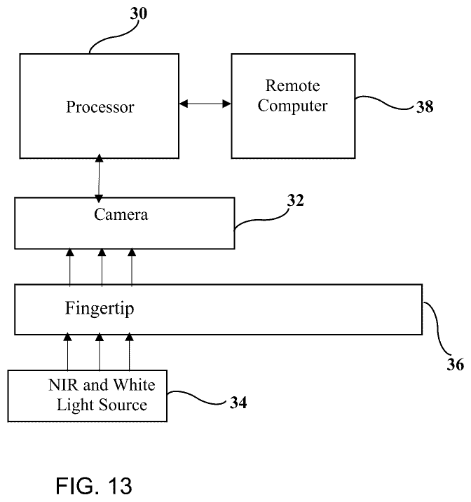

Referring now to FIG. 13, a block diagram of a device or system of devices for analyzing an object of interest, such as a finger, in accordance with the present disclosure is shown. The system includes a processor 30 which is in communication with a camera 32 and a light source 34. In operation the light source is activated, either by the processor or individually by, for example, input from a user or caregiver, and is positioned to shine light on the object of interest 36, which in the system described here is the finger 36. The camera 32 takes a series of pictures of the finger 36, which are preferably video but could, in some cases, be still photographs acquired in sufficiently quick succession to enable reproduction of a PPG signal, as described above. The image data is provided to the processor 30 which can either process the image data, as described below, or optionally transmit the data to a remote computer system 38 for analysis. The processor 30, camera 32, and light 34 can be part of a single device, which can be produced specifically for the application, but can also be a smart phone, laptop, tablet, or other devices having the described equipment and capable of providing light on an object to be evaluated and to acquire images of the object. The processor, camera, and light can all also be provided as separate components. The remote computer system 38 can, for example, be a cloud computer system or other types of wired or wireless networks. As described more fully below, the system can be used to evaluate hemoglobin by processing the frames of the image data and applying a trained machine learning model. Although not shown here, the processor can be further connected to various user interfaces, including a display, keyboard, mouse, touch screen, voice recognition system, or other similar devices.

In one example, image data can be captured using a personal electronic device containing processor 30, and camera 32, and the data transferred through a communications network to the remote computer or server 38 using secure communications for processing. For example, video images can be acquired with a smart phone, and a mobile application (app), such as an Android or iOS-based application, and sent to a cloud server 38 through the internet. A software application can be stored on the hand-held device and used to capture, for example, a 10-second fingertip video with the support of the built-in camera and a near infrared LED device adapted to provide illumination on a finger. The remote computer 38 can provide user authentication, video processing, and feature extraction from each video, as described above. Other methods of communicating to a remote computer can include, for example, communications via wired or wireless networks, cellular phone communications, Bluetooth communications, or storing data on a memory device and transferring the information to the remote computer through a memory drive or port.

A mobile application can store data useable by the camera 32 to monitor the position of the user's finger for appropriate placement, and activate an indicator, such as a light, or a speaker, to provide a signal to the user when the finger is appropriately positioned. The camera can also compare to stored data to evaluate whether the finger is sufficiently motionless to acquire data with the camera, and whether the finger is applying normal pressure. A video recording process can be automatically started by the mobile application when the user's finger is appropriately positioned so that user doesn't have to activate the video recording button, and stopped after a pre-determined period of time, such as a 10-second duration. The application can communicate with and automatically transfer video to the remote computer 38 or ask the user to affirm whether they are ready to transmit the data. Based on available bandwidth, the entire video can be transferred at one time. Alternatively, portions of the video can be iteratively sent to the remote computer 38. Communications through a USB port connection, Bluetooth, or other wired or wireless system can also be used with corresponding communications devices associated with the light device 34 to activate lighting.

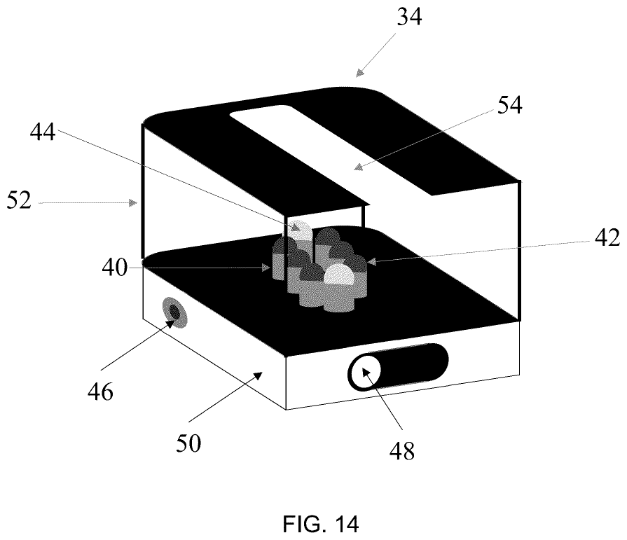

The light source 34 can be an LED associated with the device, and video can be acquired using the built-in camera in the equipment. In alternative embodiments, a specific NIR device, such as a printed circuit board can be provided (See, for example, FIGS. 3A and 3B). Referring now to FIG. 14, as discussed above, the light source 34 can have, for example, a plurality of LEDs 40 emitting light at a wavelength of 850 nm, and another plurality of LEDs 42 emitting light at a wavelength of 1070 nm. Other wavelength variations within the spectrum range of 650 nm to 1100 nm are also possible. For example, wavelengths responsive to hemoglobin can be used in a range between 800 and 950 nm. Wavelengths responsive to plasma can be in the range of 950 nm-1100 nm, with a peak response at around 1000 nm. In one embodiment, to provide a sufficient amount of light, 6 LEDs of 140 mW were used for each wavelength. One or more white lights 44 can also be provided on the board. A battery, such as a rechargeable battery, can be provided to power the LEDs. A charging point 46 for charging the battery can be included, along with a three-way or on/off switch 48. Although a single board is illustrated here, in some applications, the LEDS of specific wavelengths can be provided on two separate devices or boards, one adapted to provide NIR light responsive to plasma, and a second adapted to provide NIR light responsive to hemoglobin. In one embodiment, six 850 nm LEDs were used to provide light responsive to hemoglobin and six 1070 nm LEDs were used to provide light responsive to plasma. Two white LEDs were used to illuminate the finger during acquisition of images. This configuration was shown to be particularly successful in providing an accurate reading of hemoglobin. A similar configuration using 950 nm light also provided reasonably accurate results.

Referring still to FIG. 14, the LEDs 40, 42, and 44 are preferably mounted to a printed circuit board that can be provided in a housing 50. The charging point 46, switch 48, and battery can also be mounted in the housing 50. A light restrictive enclosure 52, which encloses the LEDs, is mounted to the housing 50, and comprises a slot 54 sized and dimensioned to receive a finger illuminated by the LEDs 40, 42, and 44. The shape of the upper layer of the enclosure 52 enables positioning a finger adjacent the board for illumination. In particular, the enclosure 52 is dimensioned to cause the dorsal area of the finger to touch the LEDs 40 and 42, and video can be captured from the opposing ventral side of the finger. Although the sides of the enclosure 52 are illustrated as open to enable viewing the LEDs, the sides of the enclosure are typically closed to prevent ambient illumination from interfering with the LEDs. The enclosure is preferably black in color, and can further be constructed of a material selected to minimize light interference from outside of the enclosure. Although a box shape is illustrated here, the shape of the enclosure is not limited to box-like enclosures, but can include, for example, a round or oblong profile sized to receive a finger, or other types of enclosures. Further, although three LEDs of each wavelength are illustrated here, the number of LEDs is not intended to be limiting. Various numbers of LEDs can be used. As described above, it has been shown experimentally that six or more LEDs of each wavelength provide improved results. Further, although LEDs of two different wavelengths are illustrated in the LED device here, LEDs 40 and 42 can be provided in separate LED devices. Where LEDs of both wavelengths are provided, the switch 48 can be a three way switch, switching between LEDs 42, LEDs 44, and an off position. When LEDs of one wavelength are provided in the enclosure, the switch 48 can be a two way on/off switch. In some applications, the LED device can be coupled to a camera, video camera, or a handheld device including a camera such as a smartphone, tablet, laptop, or similar device using brackets, straps, fasteners, adhesives, or other such devices.

Alternatively, the light 34 can be coupled directly to the user's finger, such as the index finger, using coupling devices including hook and loop fasteners, adhesives, tie straps, clastic bands, or similar elements. In some application, the light 34 device may be curved or otherwise formed specifically to engage a finger. The light 34 device may also include coupling elements enabling coupling of the device to a cellular phone or other device containing the processor 30 or to a camera 32.

The system can perform the hemoglobin level prediction at a local processor, such as the processor 30, or at a remote computer 38, which can be, for example, a cloud-based device. The cloud computing system can be HIPAA (Health Insurance Portability and Accountability Act) compliant or otherwise secured to address security and privacy issues, such as protected health information (PHI), to protect the stored database from unauthorized access, and data breach.

It should be understood that the methods and apparatuses described above are only exemplary and do not limit the scope of the invention, and that various modifications could be made by those skilled in the art that would fall under the scope of the invention. For example, although specific hardware configurations are described above, it will be apparent that a number of variations are available. Images of an illuminated finger could, for example, be acquired by a camera and transferred directly to a computer through hard wired or wireless links, or through transportable memory storage such as an SD card, USB flash drive, or other device. As described above, processing to analyze the hemoglobin content of a PPG signal acquired from a series of images or video can be performed by a local processor or computer, or at a remote location, such as a cloud device, as described above. Various off the shelf handheld devices, including smartphones and cellular phones that include an on-board camera and a processor can be used in the process described above. However, a device constructed specifically for this purpose can also be used.

To apprise the public of the scope of this invention, the following claims are made:

1. A method for non-invasive analysis of a hemoglobin level, the method comprising the following steps:
illuminating a finger of a subject with a near infrared light of a wavelength responsive to blood hemoglobin;
acquiring a first time series of images of the finger of the subject while illuminated by the near infrared light of a wavelength responsive to blood hemoglobin to capture at least one complete detailed Photoplethysmography (PPG) cycle representative of blood hemoglobin;
illuminating the finger of the subject with a near infrared light of a wavelength responsive to blood plasma; and
acquiring a second time series of images of the finger of the subject while illuminated with the near infrared light of a wavelength responsive to blood plasma to capture at least one complete detailed PPG cycle representative of plasma;
identifying at least one feature in the PPG cycle representative of blood hemoglobin;
identifying at least one feature in the PPG cycle representative of blood plasma;
providing the identified feature representative of blood hemoglobin and the feature representative of blood plasma to a predictive model adapted to identify a hemoglobin level as a function of the features.

2. The method of claim 1, wherein the steps of acquiring a first time-based series of images and acquiring a second time-based series of images comprise acquiring a first and a second video, and wherein the near infrared light responsive to blood hemoglobin has a wavelength of between 800 and 950 nm and the near infrared light responsive to plasma has a wavelength of 1070 nm.

3. The method of claim 2, wherein the near infrared light responsive to blood hemoglobin has a wavelength of 850 nm.

4. The method of claim 1, further comprising the step of identifying at least one feature in each of the PPG cycles, the feature used to determine the hemoglobin level.

5. The method of claim 1, further comprising the step of calculating a ratio of the PPG signal of the first time-based series of images of a blood flow illuminated with a near infrared light responsive to blood hemoglobin, to the second time-based series of the images of a blood flow illuminated with a near infrared light responsive to blood plasma.

6. The method of claim 4, wherein the feature comprises at least one of a relative augmentation of a PPG, an area under the systolic peak; an area under a diastolic peak, a slope of the systolic peak, a slope of the diastolic peak, a relative timestamp value of the peak, a normalized PPG rise time, a pulse transit time (PTT), a pulse shape, or an amplitude.

7. The method of claim 2, further comprising the step of separating the video into frames, each frame comprising an image.

8. The method of claim 1, wherein the step of processing comprises analyzing the PPG signals using a prediction model constructed using a support vector machine regression.

9. The method of claim 1, wherein the near infrared light responsive to blood plasma has a wavelength of 1070 nm.

10. The method of claim 1, wherein the near infrared light responsive to hemoglobin has a wavelength of 850 nm.

11. The method of claim 1, wherein the step of generating a time series signal for each of the first and second time-based series of images comprises acquiring red green blue (RGB) digital images of a blood flow, and the step of subdividing each image into a plurality of blocks further comprises the steps of:
subdividing each image into a plurality of blocks further comprising a defined number of pixels;
calculating a mean intensity value for the red pixels in each block;
generating the time series signal identifying each image in the series versus an average value of a block; and subsequently
identifying at least one PPG signal in each time series.

12. The method of claim 11, further comprising the steps of filtering the data in each of the frames to identify PPG signals.

13. The method of claim 11, further comprising the step of sampling the images at the Nyquist frequency.

14. The method of claim 11, further comprising the step of identifying a plurality of PPG signals in each time series.

15. The method of claim 1, further comprising the steps of:
calculating a ratio of the at least one feature in the PPG cycle representative of blood hemoglobin to the at least one feature in the PPG cycle representative of blood plasma; and
providing the ratio to the predictive model, wherein the predictive model is configured to identify a hemoglobin level as a function of the ratio.

16. The method of claim 1, further comprising the step of illuminating the finger within in an enclosure made of a material selected to minimize interference from ambient light.

17. The method of claim 1, further comprising the step of illuminating the finger of the subject with a white light.

18. A method for non-invasively analyzing blood hemoglobin levels, comprising the following steps:
acquiring a time-based series of images of a finger ventral pad-tip illuminated from the dorsal side of the finger with a near infrared light responsive to blood hemoglobin, and white light;
acquiring a second time-based series of images of the finger ventral pad-tip illuminated from the dorsal side of the finger with a near infrared light responsive to blood plasma, and white light;
dividing images in each of the first and second time-based series into groups of blocks;
generating time series signals from each block;
identifying at least one Photoplethysmography (PPG) cycle from each of the time series signals, including a systolic peak and a diastolic peak; and
processing the PPG cycles to determine blood hemoglobin levels.

19. The method of claim 18, wherein the near infrared light responsive to blood hemoglobin has a wavelength of between 800 and 950 nm and the near infrared light responsive to plasma has a wavelength of 1070 nm.

20. The method of claim 18, wherein the step of processing comprises analyzing the PPG signals using a prediction model constructed using a support vector machine regression.
